(19) United States
(12) Patent Application Publication
(10) Pub. No.: US 2005/0244410 A1
(43) Pub. Date: Nov. 3, 2005

(54) Toll-like receptor 9 effector agents and uses thereof

(76) Inventors: Ashlyn Bassiri, Ardmore, PA (US); Anuk Das, Wayne, PA (US); Susan Dillon, Bryn Mawr, PA (US); Karen Duffy, Trappe, PA (US); Jonathan Seideman, Southampton, PA (US); M. Lamine Mbow, King of Prussia, PA (US); Lars Karlsson, Lajolla, CA (US); Siquan Sun, San Diego, CA (US); Jian Zhu, San Diego, CA (US); Mark Cunningham, Kennett Square, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/835,475

(22) Filed: Apr. 29, 2004

(51) Int. Cl. A61K 39/395; A61K 48/00

(52) U.S. Cl. 424/144.1; 514/44

(57) ABSTRACT

Cell surface TLR9 and TLR9 ligand binding agents are disclosed. The binding agents include antibodies and other proteins. The binding agents are useful as therapeutics, diagnostics or research reagents.

### CROSS-REFERENCE TO RELATED APPLICATION

This application claims the benefit of U.S. Provisional Application No. 60/466,341, filed Apr. 29, 2003.

### FIELD OF THE INVENTION

This invention relates to cell surface toll-like receptor 9 (TLR9) effector agents such as TLR9 receptor binding agents and TLR9 ligand binding agents and their use in modulating an immune response.

### BACKGROUND OF THE INVENTION

The immune system is armed with the means to discriminate between self and non-self antigens. To this end, the immune system has evolved a series of pattern-recognition receptors to identify invading pathogens and initiate the host immune response. The toll-like family of receptors function in this fashion to activate both the innate and the adaptive arms of the immune response (Janeway and Medzhitov, Ann. Rev. Immunol. 20: 197-216, (2002)). Mammalian toll-like receptors (TLRs) were cloned based on sequence homology to the Drosophila toll gene which plays a critical role in immunity to infection with Aspergillus fumigatus (Lemaitre et al., Cell 86: 973-983, (1996); Medzhitov et al., Nature 388: 394-397, (1997); Rock et al., Proc. Natl. Acad Sci. (USA) 95: 588-593, (1998)). It has been demonstrated that using a constitutively active form of human Toll resulted in NF-Kβ activation and upregulation of B7-1 as well as IL-1, IL-8, and IL-6 cytokine message, suggesting a role for TLRs in innate and adaptive immunity (Medzhitov et al., Nature 388: 394-397, (1997)). At present, eleven TLR family members have been identified in humans and nine in mice.

TLR9 has been identified as the receptor for the unmethylated CpG dinucleotides found in bacterial but not human DNA (Hemmi et al., Nature 408: 740-745, (2000); Krieg et al., Nature 374: 546-549, (1995)). Expression profiling revealed TLR9 mRNA or protein in B cells and plasmacytoid dendritic cells (Bauer et al., Proc. Natl. Acad. Sci. (USA) 98: 9237-9242, (2001); Krug et al., Eur. J. Immunol. 31: 3026-3037, (2001). Using synthetic CpG oligonucleotides (ODN) for TLR9 stimulation/ligation, it was found that CpG-ODN could act in an adjuvant fashion (Sun et al., J. Exp. Med. 187: 1145-1150 (1998); Lipford et al., Eur. J. Immunol. 27: 2340-2344, (1997); Chu et al., J. Exp. Med. 186: 1623-1631 (1997)) to stimulate cytokine production (Klinman et al., Proc. Natl. Acad. Sci. (USA) 93: 2879-2883, (1996)) and mediate dendritic cell maturation (Hartmann et al., Proc. Natl. Acad. Sci. (USA) 96: 9305-9310, 1999; Bauer et al., J. Immunol. 166: 5000-5007, (2001). Furthermore, it was found that cells from TLR9 deficient mice did not proliferate or secrete cytokines in response to CpG stimulation, and overall, the mice were resistant to lethal CpG-induced shock (Hemmi et al., supra).

Conflicting data has been reported as to whether TLR9 can be expressed at the cell surface, despite it sharing significant homology with other members of the TLR family including putative intracellular, extracellular, and transmembrane domain sequences (Du et al., Eur. Cytokine Netw. 11:362-371, (2002); Hemmi et al., supra).

Prior to the discovery of TLR9, studies using fluorescently labeled CpG-ODN to stimulate macrophage cell lines revealed that ODNs were rapidly taken up into the endosomal compartment with minimal localization at the plasma membrane (Hacker et al., EMBO J. 17:6230-6240, (1998)). In primary cell assays, immobilized CpG-ODN that could not be internalized were used to investigate the potential of a cell-surface receptor capable of mediating CpG triggered stimulation. While one group found that murine B cells failed to become activated when cultured with these CpG-ODN (Krieg et al., supra), another group found that immobilized CpG-ODN induced human B cell proliferation and Ig secretion comparable to free CpGs (Liang et al., J. Clin. Invest. 98:1119-1129, (1996); Liang et al., J. Immunol. 165:1438-1445, (2000)).

Following the discovery of TLR9 and the identification of the receptor-ligand relationship of TLR9 for CpG dinucleotides, (Hemmi et al., supra; Du et al., supra) the cellular localization of TLR9 still remained unclear. Chuang et al. in J. Leukoc. Biol. 71:538-544, (2002) using murine TLR9, generated data suggesting cell surface expression on transfected human HEK293 cells. These authors transiently transfected the cell line with TLR9-Flag, and found expression of Flag at the cell surface using an anti-Flag antibody, thereby suggesting that TLR9 may also be at the cell surface. The use of this artificial system resulting in TLR9 overexpression in transformed cell lines does not evaluate the physiologic localization of TLR9 in primary immune cells.

Similar results were found by Takeshita et al. (J. Immunol. 167:3555-3558, (2002)) using human TLR9 transiently transfected HEK 293 cells. These studies were criticized as the endogenous leader sequence of TLR9 (comprised of the first 26 N-terminal amino acids) had been replaced by the authors with a heterogeneous leader sequence derived from the IgK gene (Ahmad-Nejad et al., Eur. J. Immunol. 32:1958-1968, (2002)).

To further address the issue of TLR9 localization, Ahmad-Nejad et al., supra, generated a murine anti-TLR9 mAb directed toward the extracellular domain of human TLR9. Using this mAb to stain a permeablized cell line, the authors reported intracellular TLR9 expression but not cell-surface expression following IFN-γ treatment.

TLR9 stimulation has been recognized as having an important role in activating both innate and adaptive immune responses. These responses play a role in autoimmune diseases, inflammatory diseases and sepsis as well as adjuvant and anti-tumor effects. Accordingly, a need exists for antagonistic or agonistic agents that can modulate TLR9 biological activity.

### BRIEF DESCRIPTION OF THE DRAWINGS

FIG. 1 shows representative flow cytometry data for cell surface TLR9⁺ cells in tonsillar cell populations.

FIG. 2 shows representative flow cytometry data for cell surface TLR9⁺ cells in PBMC populations.

FIG. 3 shows a blockade of TLR9 staining using a TLR9 peptide.

FIG. 4 shows a control peptide does not prevent TLR9 staining.

FIG. 5 shows CpG-dependent binding of a TLR9 extracellular domain to CpG-ODN.

### SUMMARY OF THE INVENTION

One aspect of the invention is a method of modifying antigen presenting cell function in a patient in need thereof comprising administering to the patient a cell surface TLR9 binding agent that specifically binds to human TLR9 in an amount effective to modify antigen presenting cell function in the patient.

Another aspect of the invention is a method of identifying TLR9 binding agents comprising the steps of contacting MHCII⁺CD19⁺ or MHCII⁺CD19⁻ primary human cells expressing TLR9 on their surface with a putative binding agent; measuring the binding of the putative binding agent to the cell surface and the effect on TLR9 biological activity; and identifying TLR9 binding agents affecting TLR9 biological activity.

Another aspect of the invention is a method of modifying antigen presenting cell function in a patient in need thereof comprising administering to the patient a TLR9 ligand binding agent in an amount effective to modify antigen presenting cell function in the patient.

Another aspect of the invention is a TLR9 ligand binding agent comprising residues 1 to 260 of the extracellular domain of human TLR9 protein, a fragment thereof or its mature form.

Other aspects of the invention are a TLR9 ligand binding agent comprising residues 1 to 260 of human TLR9 protein extracellular domain, a fragment thereof or its mature form fused to a fusion partner and its use in identifying TLR9 binding agents.

### DETAILED DESCRIPTION OF THE INVENTION

All publications, including but not limited to patents and patent applications, cited in this specification are herein incorporated by reference as though fully set forth.

The terms “agonist” and “agonistic” as used herein refer to or describe a molecule that is capable of, directly or indirectly, substantially inducing, promoting or enhancing TLR9 biological activity or TLR9 receptor activation.

The terms “antagonist” or “antagonistic” as used herein refer to or describe a molecule that is capable of, directly or indirectly, substantially counteracting, reducing or inhibiting TLR biolocial activity or TLR9 receptor activation.

The term “antibodies” as used herein is meant in a broad sense and includes immunoglobulin or antibody molecules including polyclonal antibodies, monoclonal antibodies including murine, human, humanized and chimeric monoclonal antibodies and antibody fragments.

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄.

The term “antibody fragments” means a portion of an intact antibody, generally the antigen binding or variable region of the intact antibody. Examples of antibody fragments include Fab, Fab′, F(ab′)₂ and Fv fragments, diabodies, single chain antibody molecules and multispecific antibodies formed from at least two intact antibodies.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

The term “mimetibody” as used herein means a protein having the generic formula (I):
(V1(n)-Pep(n)-Flex(n)-V2(n)-pHinge(n)-CH2(n)-CH3(n))(m) (I)
where V1 is at least one portion of an N-terminus of an immunoglobulin variable region, Pep is at least one bioactive peptide that binds to cell surface TLR9, Flex is polypeptide that provides structural flexablity by allowing the mimetibody to have alternative orientations and binding properties, V2 is at least one portion of a C-terminus of an immunoglobulin variable region, pHinge is at least a portion of an immunoglobulin variable hinge region, CH2 is at least a portion of an immunoglobulin CH2 constant region and CH3 is at least a portion of an immunoglobulin CH3 constant region, where n and m can be an integer between 1 and 10. A mimetibody mimics properties and functions of different types of immunoglobulin molecules such as IgG₁, IgG2, IgG3, IgG4, IgA, IgM, IgD and IgE. A mimetibody of the present invention affects TLR9 biological activity through binding to cell surface TLR9.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256: 495 (1975). Chimeric mAbs containing a light chain and heavy chain variable region derived from a donor antibody (typically murine) in association with light and heavy chain constant regions derived from an acceptor antibody (typically another mamammlian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl Acad Sci (USA), 86: 10029-10032, (1989) and Hodgson et al., Bio/Technology, 9: 421, (1991).

Fully human mAbs lacking any non-human sequences can be prepared from human immunoglobulin transgenic mice by techniques referenced in, e.g., Lonberg et al., Nature 368: 856-859, (1994); Fishwild et al., Nature Biotechnology 14: 845-851, (1996)′ and Mendez et al., Nature Genetics 15: 146-156, (1997). Human mAbs can also be prepared and optimized from phage display libraries by techniques referenced in, e.g., Knappik et al., J. Mol. Biol. 296: 57-86, (2000) and Krebs et al., J. Immunol. Meth. 254: 67-84, (2001).

The term “TLR9 biological activity” or “TLR9 receptor activation” as used herein refers to any activation of the innate or adaptive arms of the immune response or any activities occurring as a result of ligand binding to cell surface TLR9.

The present invention relates to agents that can bind specifically to TLR9 on mammalian cell surfaces. The cell surface TLR9 binding agents are useful as agonists or antagonists to modify the function of TLR9 located on the cell surface. These binding agents are useful as research reagents, diagnostic reagents and potential therapeutic agents. In one embodiment of the invention, the agents bind specifically to TLR9 on human cell surfaces.

In particular, the invention relates to the use of the agonists or antagonists to modify the TLR9 biological activity of distinct subsets of MHC ClassII⁺CD19⁺ (MHCII⁺CD19⁺) human cells such as MHCII⁺CD19⁺CD123^{low} and MHC ClassII⁺CD19⁻ human cells such as MHCII^{low}CD19⁻CD123^{bright} and MHCII^{low}CD19⁻CD123^{low}. These subsets can be antigen presenting cells such as B cells or dendritic cells. Cell surface TLR9 agonists and antagonists include, but are not limited to, any antibody, fragment or mimetibody; any soluble receptor, fragment or mimetic; or any small organic molecule; or any combination of the foregoing. TLR9-specific mAbs are included as one type of such an agonist or antagonist.

Anti-TLR9 mAbs can be generated in normal mice using standard hybridoma technology techniques (Kohler et al., supra) well known to those skilled in the art. Briefly, separate groups of mice are immunized with human TLR9 (SEQ ID NO: 1) or a fragment such as the extracellular domain (residues 1 through 819 of SEQ ID NO: 1) emulsified in complete Freund's adjuvant (CFA). Each mouse receives 25 μg of the immunogen in CFA followed by an equal amount of the immunogen in incomplete Freund's adjuvant two weeks later. Alternatively, mice can receive two injections (two weeks apart) of plasmid DNA encoding human TLR9 or a fragment thereof, such as the extracellular domain (10 μg/mouse), followed by a booster injection with human TLR9 protein or a fragment thereof, such as the extracellular domain.

Three days prior to B cell fusion, protein or DNA-immunized mice are given an intravenous injection of the immunogen in phosphate-buffered saline (PBS) at 15 μg immunogen per mouse. Spleens from immunized mice are harvested and B cell fusion carried out using the methods of Kohler et al., (supra). Fused cells are selected using medium containing hypoxanthine-aminopterin-thymidine (HAT) and wells are screened for the presence of anti-TLR9 antibodies by enzyme-linked immunosorbent assay (ELISA). Positive wells are expanded and cloned by limiting dilution.

Another aspect of the invention is a method of identifying TLR9 binding agents comprising the steps of contacting MHCII⁺CD19⁺ or MHCII⁺CD19⁻ primary human cells expressing TLR9 on their surface with a putative binding agent; measuring the binding of the putative binding agent to the cell surface and the effect on TLR9 biological activity; and identifying TLR9 binding agents affecting TLR9 biological activity. The TLR9 binding agents that can be identified by this method of the invention include small organic molecules, oligonucleotides, nucleic acids, peptides, antibodies and other proteins.

The present invention also relates to TLR9 ligand binding agents and their use. TLR9 ligand binding agents function as antagonists by binding TLR9 ligands, thereby preventing the ligands from binding to TLR9 located on the cell surface. These binding agents are useful as research reagents, diagnostic reagents and potential therapeutic agents. In one embodiment of the invention, the agents bind specifically to human TLR9 ligands.

TLR9 ligand binding agents of the invention include residues 1 to 260 of the extracellular domain of human TLR9 protein, a fragment thereof or its mature form lacking a leader sequence. Also included are fusion proteins where residues 1 to 260 of the extracellular domain of human TLR9 protein, a fragment thereof or the mature form are fused to a fusion partner such as the Fc portion of an immunoglobulin molecule or a mimetibody. An exemplary TLR9 ligand binding agent of the invention is a fusion construct including residues 1 to 260 of the extracellular domain of human TLR9 protein fused to an IgG1 Fc region having the amino acid sequence shown in SEQ ID NO: 2.

As mentioned above, one embodiment of the TLR9 ligand binding agents of the invention is the mature form of the 260 residue extracellular domain of human TLR9 protein as well as a fusion construct containing the mature form. The mature secreted form of this extracellular domain fragment will lack the signal sequence. The signal sequence cleavage site for this extracellular domain fragment is predicted to be at residue 25 of SEQ ID NO: 2. However, it will be recognized by those skilled in the art that the actual signal sequence cleavage site can vary from the predicted cleavage site. Thus, another exemplary TLR9 ligand binding agent of the invention is a fusion construct including the mature form of the 260 residue extracellular domain of human TLR9 protein fused to an IgG1 Fc region. One example is the fusion protein having the amino acid sequence shown in SEQ ID NO: 11.

The exemplary ligand binding agents of the invention can be expressed using standard recombinant protein expression platforms, e.g., mammalian cell expression systems, and utilize either stable cell lines or transient transfection production procedures.

The TLR9 ligand binding agents of the invention can also be used in a method of identifying TLR9 binding agents by contacting a TLR ligand binding agent with a putative TLR9 binding agent; measuring the binding of the putative TLR9 binding agent to the TLR9 ligand binding agent and the effect on TLR9 biological activity; and identifying TLR9 binding agents affecting TLR9 biological activity. The TLR9 binding agents that can be identified by this method of the invention include small organic molecules, oligonucleotides, nucleic acids, peptides, antibodies and other proteins.

TLR9, like other TLRs, may consist of TLR heterodimers or as yet unidentified adapter molecules. Therefore, primary cell populations expressing TLR9 in its natural form, unlike TLR9 transfected cell lines, represent an ideal tool for the selection of agonistic or antagonistic TLR9-specific mAbs. Without the use of primary cell populations expressing TLR9 for mAb screening, it is possible that mAbs directed toward significant epitopes of TLR9 would be missed. For screening purposes, primary cells would be incubated with hybridoma supernatants or purified hybridoma-generated mAbs with or without bacterial DNA. Cytokine production, or lack thereof would be used to identify both agonistic and antagonistic TLR9-specific mAbs.

A cell surface TLR9 agonist is useful for treating a number of mammalian disease states including, but not limited to, pathologic conditions related to bacterial, viral, parasitic, or fungal infections particularly Herpes simplex virus (HSV), Human papilloma virus (HPV) and Chlamydia; treatment and/or augmentation of other therapies used to treat cancer; and in treatment of pathologies associated with allergic responses such as asthma.

While not wishing to be bound to any particular theory, it is thought that TLR9 agonists will be useful as an adjuvant in all types of infections (bacterial, viral, parasitic, and fungal). Further, given the effectiveness of the TLR9 agonist CpG to treat genital infections such as herpes simplex virus (Pyles et al., J. Virol. 76: 11387-11396, (2002)), a TLR9 agonist is likely to be effective in treating a variety of genital infections including HSV, HPV and Chlamydia. TLR9 agonists could also be used topically to prevent or treat symptoms associated with genital tract infections.

Also, again not wishing to be bound to any particular theory, TLR9 agonists will be useful to treat cancer because of their potent effects on innate immunity. TLR9 agonists will be useful either as monotherapy or in combination with cancer cytotoxics or anti-cancer mAbs since bacterial DNA has been shown to have potent anti-tumor effects (Tokunaga et al., J. Natl. Cancer Inst. 72: 955-962, (1984)) and a synthetic single-stranded DNA was also found to have anti-tumor properties (Tokunaga et al., Jpn. J. Cancer Res. 79:682-686, (1988)).

Further, and again not wishing to be bound by any particular theory, TLR9 agonists will be useful in treating diseases that have a Th2-mediated immunopathology, e.g., asthma, allergy, pulmonary fibrosis and ulcerative colitis. CpG-ODNs and immunostimulatory sequences (ISS) have been shown to prevent the development of allergic airway responses in animal models (Kline et al., J. Immunol. 160: 2555-2559, (1998)) by inducing a potent Th1 response. Therefore, TLR9 agonists are also expected to be useful in this regard. For specific desensitization to allergens, the allergen could be conjugated to the TLR9 mAb as described for ragweed-conjugated ISS (Santeliz et al., J. Allergy Clin. Immunol. 109: 455-462, (2002)). In contrast to synthetic ODN or ISS, TLR9-specific mAbs would have a longer plasma half-life and would selectively target the cell surface TLR9 molecules.

A cell surface TLR9 antagonist is useful for treating a number of mammalian disease states including, but not limited to, autoimmune disorders such as systemic lupus erythematosus, Sjögren's syndrome, Scleroderma and CREST syndrome, multiple sclerosis, Th1-cell mediated inflammatory disease, sarcoidosis, cystic fibrosis and rheumatoid arthritis, inflammatory conditions such as chronic obstructive pulmonary disease (COPD), inflammatory bowel disease and sepsis.

While not wishing to be bound to any particular theory, it is thought that TLR9 antagonists will be useful for treating the autoimmune diseases mentioned above due to the likely role of TLR9 stimulation in the aberrant activation of autoreactive B cells (Leadbetter et al., Nature 416: 603-607, (2002)) coupled with the fact that DNA methylation is known to be decreased in cells from autoimmune humans and mice (Richardson et al., Arthritis Rheum. 33: 1665-1673, (1990)). In addition, the association between infectious disease illnesses and flare-ups of multiple sclerosis can be correlated with release of bacterial DNA that binds TLR9 (Ichikawa et al., J. Immunol. 169: 2781-2787, (2002)), suggesting a potential therapeutic benefit of TLR9 antagonists in multiple sclerosis.

Also, and again not wishing to be bound to any particular theory, it it thought that TLR9 antagonists will be useful for treatment of inflammatory diseases due to the fact that blockade of the interaction between bacterial DNA and TLR9 can alleviate the Th1-driven inflammatory response during bacterial infections. For example, the appearance of certain bacterial strains in the sputum of patients with COPD is associated with disease exacerbation (Sethi et al., New Engl. J. Med. 347: 465-471, (2002)), suggesting that a TLR9 antagonist may have therapeutic benefit in inflammatory diseases such as COPD, emphysema and sarcoidosis. The potential role of bacterial species as initiators of the inflammatory process in inflammatory bowel disease support the use of TLR9-specific antagonists to block prolonged cell activation.

Further, and again not wishing to be bound to any particular theory, it it thought that TLR9 antagonists will be useful for treatment of sepsis due to the fact that release of free bacterial DNA is likely to contribute to the cytokine storm during bacterial sepsis. Therefore, a TLR9 antagonist may be more efficient in treating bacterial sepsis than targeting individual cytokines.

Because TLR9 can be expressed at the cell surface, identification of a peptide agonist may be suitable for a mimetibody approach. This approach may result in increased potency compared to agonist mAbs, CpG-ODN, or ISS, and therefore will likely require less dosing and may be less expensive than other therapies (e.g., CpG-based therapies) which target TLR9.

The discovery of surface localization of TLR9 further allows for the generation of and uses for agents binding TLR9 as targeting moieties to specifically identify, activate, or destroy cells displaying this marker on their surface. The invention therefore further describes the use of subsets of MHCII⁺CD19⁺ and MHCII⁺CD19⁻ primary human cells to identify compounds and compositions capable of specifically binding TLR9 and, more particularly, those agents capable of modifying TLR9 biological activity.

The mode of administration for therapeutic use of the binding agents of the invention may be any suitable route which delivers the agent to the host. The proteins, antibodies, antibody fragments and mimetibodies and pharmaceutical compositions of these agents are particularly useful for parenteral administration, i.e., subcutaneously, intramuscularly, intradermally, intravenously or intranasally.

Binding agents of the invention may be prepared as pharmaceutical compositions containing an effective amount of the binding agent as an active ingredient in a pharmaceutically acceptable carrier. An aqueous suspension or solution containing the binding agent, preferably buffered at physiological pH, in a form ready for injection is preferred. The compositions for parenteral administration will commonly comprise a solution of the binding agent of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the binding agent of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of a binding agent of the invention. Similarly, a pharmaceutical composition of the invention for intravenous infusion could be made up to contain about 250 ml of sterile Ringer's solution, and about 1 mg to about 30 mg and preferably 5 mg to about 25 mg of a binding agent of the invention. Actual methods for preparing parenterally administrable compositions are well known or will be apparent to those skilled in the art and are described in more detail in, for example, “Remington's Pharmaceutical Science”, 15th ed., Mack Publishing Company, Easton, Pa.

The binding agents of the invention, when in a pharmaceutical preparation, can be present in unit dose forms. The appropriate therapeutically effective dose can be determined readily by those of skill in the art. A determined dose may, if necessary, be repeated at appropriate time intervals selected as appropriate by a physician during the treatment period.

The protein, TLR9 mAb or mimetibody binding agents of the invention can be lyophilized for storage and reconstituted in a suitable carrier prior to use. This technique has been shown to be effective with conventional immunoglobulins and protein preparations and art-known lyophilization and reconstitution techniques can be employed.

The present invention will now be described with reference to the following specific, non-limiting Examples.

### EXAMPLE 1

### TLR9 Surface Expression in Tonsillar Cells

Human tonsil samples, harvested from pediatric donors, were obtained from the National Disease Research Interchange (Philadelphia, Pa.). Tissue samples were dissected into small pieces and incubated with 1 mg/ml Collagenase D (Boehringer Mannheim, Mannheim, Germany) for one hour at 37° C. Subsequently, samples were dissociated by passage through a cell strainer and then washed two times to remove the collagenase. One million cells were stained per condition for flow cytometry.

Cells were stained using a commercially available unlabelled mouse anti-human TLR9 mAb (Imgenix, San Diego, Calif.) followed by a goat anti-mouse IgG F(ab′)₂-Cy5 (Jackson ImmunoResearch, West Grove, Pa.) detecting reagent for single color fluorescence. However, use of this secondary detecting reagent prevented multi-parameter staining with other mouse anti-human lineage marker mAbs. Therefore, for multi-parameter staining, the mouse anti-human TLR9 mAb was directly conjugated with allophycocyanin (APC) (Molecular Probes Eugene, Oreg.) and used in combination with other cell surface marker mouse anti-human mAbs: MHCII-PerCP, CD123-PE, and CD19-FITC all purchased from BD-Pharmingen (San Diego, Calif.). In every case, the percentage of TLR9 positive cells determined by directly conjugated mAb staining mirrored that found with the indirect two-step staining process (n=4). Mouse anti-human IgG (BD-Pharmingen) was used with the secondary detecting reagent or labeled with APC for use as an isotype control. Cells were read on a BD FACSCalibur™ System and samples were analyzed using Cellquest™ Pro software (BD Biosciences, San Jose, Calif.).

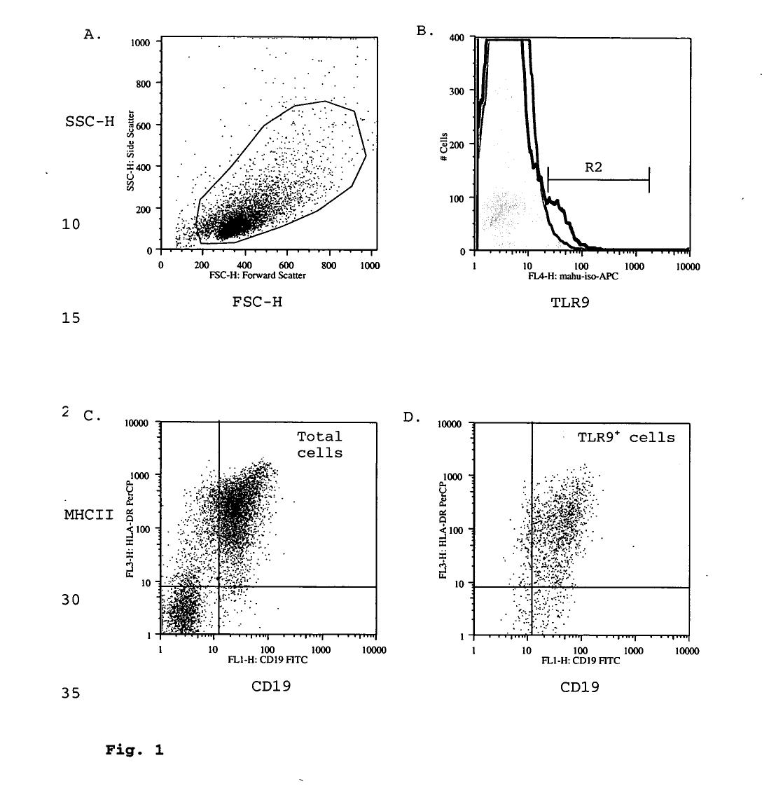

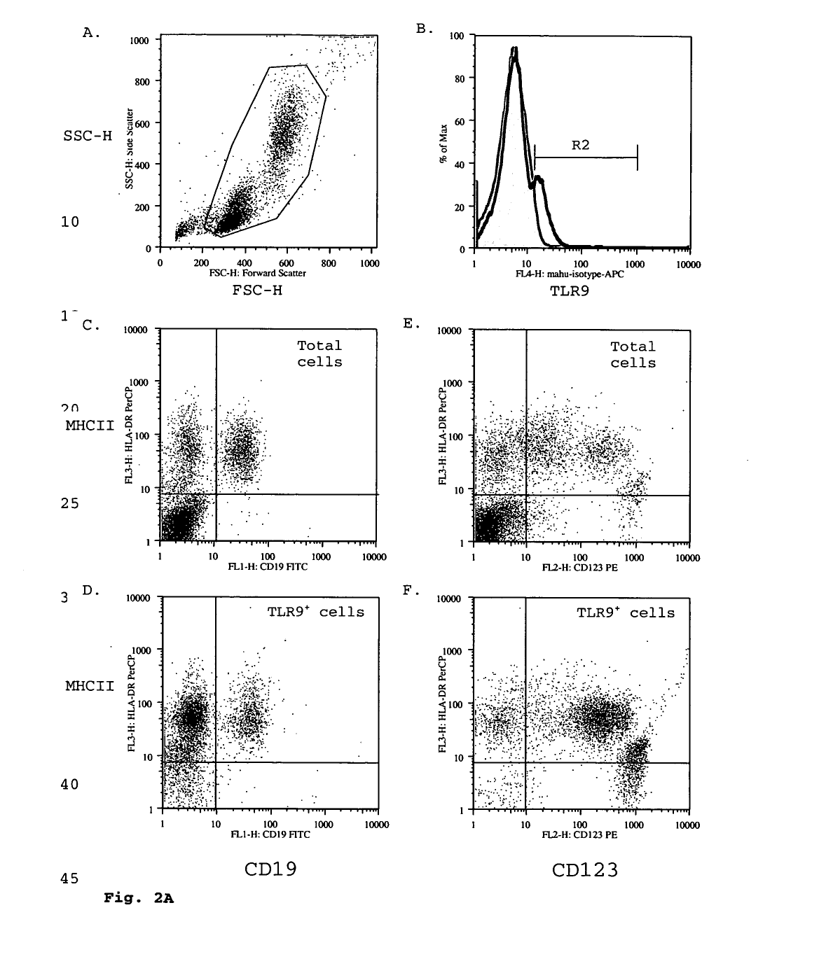

In FIG. 1, a dot plot depicting forward scatter (FSC-H) and side scatter (SSC-H) of the tonsil samples is shown in (A). A histogram displaying TLR9 staining (bold open line where staining is marked by R2) relative to control levels of staining with an isotype control mAb (gray shaded area) is shown in (B). Comparative dot plot flow staining for the total cell population (C) vs. the TLR9⁺ cell population (D) (R2 gate) for MHC ClassII and CD19 levels to elucidate the cell-surface phenotype of the TLR9⁺ cells.

Using flow cytometry on unpermeablized cells, TLR9 staining was found on the cell surface of a subset of live cell gated tonsil cells. Six different experiments were performed and a summary of the data is shown in Table 1. The percentage of TLR9⁺ cells varied from 2.2 to 9.5% of live gated tonsil cell preparations. Clearly, variability exists among the different samples in the proportion of cells that are positive for TLR9. This likely represents individual donor variation, as samples were harvested from patients with varying degrees of tonsillitis and/or tonsil hypertrophy. To determine which cell populations in the tonsil samples exhibited surface expression of TLR9, multi-parameter staining with the pan antigen presenting cell marker MHC Class II (MHCII) and the pan B cell marker CD19 was performed. Table 1 shows summary data from flow cytometric staining of MHCII and CD19 expression on TLR9⁺ live gated cells. In these experiments it was found that regardless of the overall percentage of TLR9⁺ cells, greater than 95% of the TLR9⁺ cells were B cells as indicated by their CD19 expression. The remainder of the TLR9⁺ population expressed a phenotype of MHCII^{low}CD19⁻. Overall, these data identify B cells as the primary cell population displaying TLR9 surface expression in the tonsil. Although previous data had suggested that B cells could express TLR9 mRNA (Bauer et al., Proc. Natl. Acad. Sci. (USA) 98: 9237-9242, (2001); Krug et al., Eur. J. Immunol. 31: 3026-3037, (2001)), the data presented here provide direct visual evidence for TLR9 cell surface protein expression on human primary tonsil cell populations.

TABLE 1
Relative frequency of TLR9 positive cells in tonsil
samples. The proportion of live cell gated TLR9⁺ tonsil cells is
shown (column one) relative to the isotype control (column two).
Within those TLR9⁺ populations, the proportion of cells that are
MHCII⁺CD19⁺ (column three) or MHCII^{low}CD19⁻ (column four)
are shown.

| 1 | 2 | 3 | 4 |
|---|---|---|---|
| Total | Isotype | TLR9⁺ | TLR9⁺ |
| TLR9⁺ | control | MHCII⁺CD19⁺ | MHCII^{low}CD19⁻ |
| 2.2% | 0% | ND | ND |
| 9.5% | 1.8% | ND | ND |
| 3.8% | 0% | 95.9% | 4.1% |
| 9.2% | 0% | 96.2% | 3.8% |
| 2.2% | 0.5% | 96.4% | 3.6% |
| 2.7% | 1.2% | 96.1% | 3.9% |

### EXAMPLE 2

### TLR9 Surface Expression on Peripheral Blood Mononuclear Cells

Human peripheral blood mononuclear cells (PBMC) were isolated from whole blood samples using Ficoll gradient centrifugation. One million cells were stained per condition for flow cytometry.

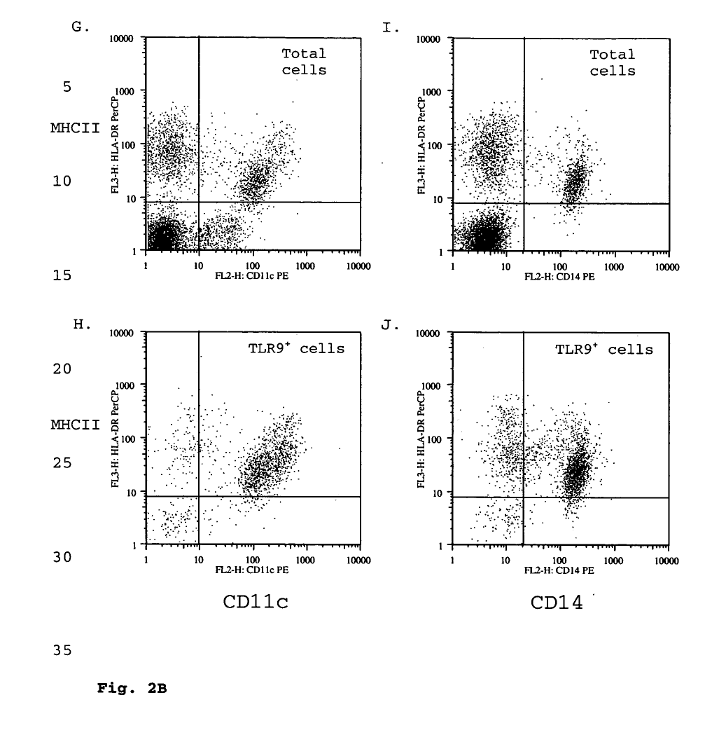

In FIG. 2, a dot plot depicting FSC-H and SSC-H of the PBMC samples is shown in (A). A histogram displaying TLR9 staining (bold open line where staining is marked by R2) relative to control levels of staining with an isotype control mAb (gray shaded area) is shown in (B). C-J show comparative dot plots of flow staining for the total cell population vs. the TLR9⁺ cell population (R2 gate) for MHCII and CD19 (C, D), CD123 (E, F), CD11c (G, H), CD14 (I, J) to elucidate the cell-surface phenotype of the TLR9⁺ cells.

In six experiments, TLR9 staining was evident on a subset of PBMC. The proportion of TLR9⁺ cells ranged from 1 to 13.3% of total live gated cells, relative to the isotype control (Table 2). To determine which cell population(s) were expressing TLR9, multi-parameter staining with MHCII, CD19, and CD123 was performed. CD123, also known as IL-3 receptor alpha, is expressed on a variety of cell types, and at very high levels on plasmacytoid dendritic cells (Dzionek et al., J. Immunol. 165: 6037-6046, (2000)). Because plasmacytoid dendritic cells are also CD19⁻ and MHCII^{low} (O'Doherty et al., Immunology 82: 487-493, (1994); Grouard et al., J. Exp. Med. 185: 1101-1111, (1997)), four color flow cytometric staining can be used to determine whether TLR9 is expressed on the cell surface of cells expressing markers of plasmacytoid dendritic cells. The results of four color flow cytometric staining for TLR9 population subtyping are shown in Table 2. In these experiments, it was found that sizeable populations of both MHCII⁺CD19⁺CD123^{low} wand MHCII^{low}CD19⁻CD123^{bright} cells displayed surface expression of TLR9. However, unlike tonsil cells, the majority of TLR9⁺ cells in PBMC lack CD19 expression (87% and 89%). Furthermore, a portion of the TLR9⁺ cell population is CD123^{bright} and MHCII^{low}CD19⁻, a cell surface phenotype suggestive of plasmacytoid dendritic cells (Dzionek et al., supra). The majority of the TLR9⁺ population expresses both CD11c (column 5) and CD14 (column 6). These data provide direct evidence for TLR9 cell surface protein expression on human PBMC populations.

TABLE 2
Relative frequency of TLR9 positive cells in PBMC samples.
The proportion of live cell gated TLR9⁺ PBMC is shown (column one)
relative to the isotype control (column two). Within the TLR9⁺
population, the proportion of cells that are MHCII⁺CD19⁺CD123^{low}
(column three) or MHCII^{low}CD19⁻CD123^{bright} (column four) are shown.
The relative proportions of MHCII⁺CD19-CD123^{low} expressing
either CD11c (column 5) or CD14 (column 6) are shown for experiments
5 and 6. The average percentage and standard deviation values are
shown in the last row for the populations of interest.

| | | | 3 | 4 | 5 | 6 |
|---|---|---|---|---|---|---|
| | 1 | 2 | TLR9⁺ | TLR9⁺ | TLR9⁺ | TLR9⁺ |
| | Total | Isotype | MHCII⁺CD19⁺ | MHCII^{low}CD19⁻ | MHCII⁺CD19⁻ | MHCII⁺CD19⁻ |
| | TLR9⁺ | control | CD123^{low} | CD123^{bright} | CD123^{low}CD11c⁺ | CD123^{low}CD14⁺ |
| Exp. 1 | 5.9% | 1.0% | 12.5% | 31.9% | | |
| Exp. 2 | 1.0% | 0.2% | 13.0% | 6.1% | | |
| Exp. 3 | 7.1% | 0.6% | 11.2% | 3.5% | | |
| Exp. 4 | 8.4% | 0.7% | 14.1% | 2.4% | | |
| Exp. 5 | 13.3% | 0.8% | 2.5% | 2.6% | 89.3% | 71.7% |
| Exp. 6 | 4.4% | 1.0% | 26.2% | 9.3% | 63.3% | 54.3% |
| Average | 6.7 ± 4.2 | 0.6 ± 0.4 | 13.3 ± 7.6 | 9.3 ± 11.4 | 76.3 ± 18.4 | 63.0 ± 12.3 |

### EXAMPLE 3

### LPS Mediated Up-Regulation of PBMC TLR9 Surface Expression

Whole PBMC were cultured overnight in either media alone or in media containing 10 μg/ml of bacterial lipopolysaccharide (LPS). Following culture, TLR9 levels were analyzed via flow cytometry. Prior to LPS stimulation, 4.4% of the PBMC population expressed cell-surface TLR9 (Table 2). After 18 hours in culture, 6.9% of the control PBMC cultured in media alone and 10.9% of the PBMC cultured in LPS had detectable levels of cell-surface TLR9 (Table 3). Importantly, PBMC stimulated for 18 hours with LPS expressed approximately 4-fold higher cell-surface levels of TLR9 (159.4 Mean Fluorescence Intensity (MFI)) relative to those PBMC in media alone (40.7 MFI) (Table 3). These data demonstrate that activation of PBMC with LPS results in the upregulation of TLR9 expression. These data suggest a cross-regulatory mechanism of expression for TLR4 and TLR9, both of which have ligands that are derived from bacterial components.

TABLE 3
Mean fluorescence intensity of TLR9 staining on cultured
PBMC. PBMC populations from Exp. 6 (Table II) were cultured
overnight in either media alone or media with 10 μg/ml of LPS. The
relative frequency of TLR9+ cells and the mean fluorescence
intensity of the staining are shown. Overnight culture with LPS
upregulates the level of TLR9 expression greater than 3.5-fold on
PBMC.

| | 1 | 2 | 3 | 4 |
|---|---|---|---|---|
| MAb | Isotype | TLR9 | Isotype | TLR9 |
| Culture | control | staining | control | staining |
| condition | Media | Media | LPS | LPS |
| Exp. 6 | 0.8% | 6.9% | 0.8% | 10.9% |
| PBMC post | | | | |
| culture | | | | |
| Mean | 31.6 | 40.7 | 29.0 | 159.4 |
| fluorescence | | | | |
| intensity of | | | | |
| staining | | | | |

### EXAMPLE 4

### Immunofluorescence Detection of PBMC Cell Surface TLR9

To observe visually whether the mouse anti-human TLR9 mAb was recognizing TLR9 at the cell-surface or rather inside the cell, cytospins of PBMC from the LPS stimulated cultures were made. Cytospins of PBMC stained with anti-CD19-FITC (a B cell marker) and either anti-TLR9-APC or an isotype control-APC were viewed by fluorescence microscopy. Images of individual slide fields of these cytospins were viewed and captured at 40× magnification under a wavelength of light capable of detecting FITC (green fluorescence) and under light capable of detecting APC (far red fluorescence).

CD19-FITC staining was observed on LPS stimulated PBMC cells, while no staining was detectable on those same cells with the mouse isotype control mAb labeled with APC (data not shown). Importantly, staining was observed with the mouse anti-human TLR9-APC (data not shown) on cytospins of LPS stimulated PBMC. CD19-FITC was also observed on the corresponding microscopy field (data not shown). However, TLR9 staining was observed on larger cells not found to be staining with CD19. These data are consistent with the data obtained by flow cytometry where the TLR9⁺ cells found in PBMC samples were observed to be larger cells, few of which were CD19⁺. Importantly, the TLR9 staining observed on the cytospins of the LPS stimulated PBMC cultures appeared to be at the cell-surface, a finding consistent with the flow cytometric analysis of TLR9 expression.

### EXAMPLE 5

### Specificity of the Fluorescently Conjugated TLR9 mAb

A commercially available mouse anti-human TLR9 mAb (Imgenix, San Diego, Calif.). This antibody was made by immunizing a mouse with a 15-mer peptide of TLR9 having the amino acid sequence CPRHFPQLHPDTFSHLS (SEQ ID NO: 3) conjugated to keyhole limpet hemocyanin (KLH) using standard hybridoma technology. The peptide represented residues 268-284 of human TLR9 located in the putative extracellular domain. To test the specificity of the flow cytometric staining observed with the mouse anti-human TLR9 mAb, the immunizing peptide was synthesized and compared with a control peptide (residues 31-45 of human prostate specific antigen (PSA)) having the amino acid sequence CEKHSQPWQVLVASR (SEQ ID NO: 4) for the ability to block the TLR9 staining observed by flow cytometry. The TLR9 peptide or control peptide was preincubated with the mouse anti-human TLR9 mAb or the isotype control mAb for 15 minutes prior to its addition to the PBMC preparation. Labeled PBMCs were then analyzed by flow cytometry. Flow cytometry methods, instrumentation, software, mAbs and PBMC preparations were as described in the preceding Examples.

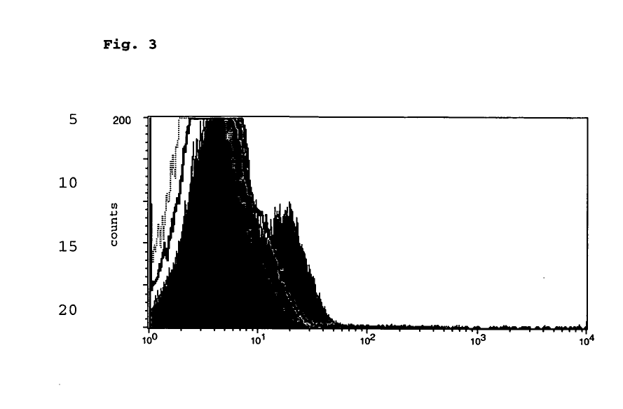

No effect on mouse isotype control staining was observed with either the TLR9 peptide or the control peptide (data not shown). Importantly, preincubation of the mouse anti-human TLR9 mAb with the TLR9 peptide reduced the level of TLR9 staining to close to background levels (those levels observed with the isotype control mAb) (FIG. 3). Histograms shown are gated on live cells and show fluorescence for the mouse anti-human TLR9 mAb (gray histogram), the mouse anti-human TLR9 mAb - preincubated with the TLR9 peptide (bold black line), and the mouse isotype-APC mAb (thin stippled line). Preincubation of mouse anti-human TLR9 mAb with a TLR9 peptide reduces the fluorescence staining observed for TLR9 to near background levels observed with the isotype control.

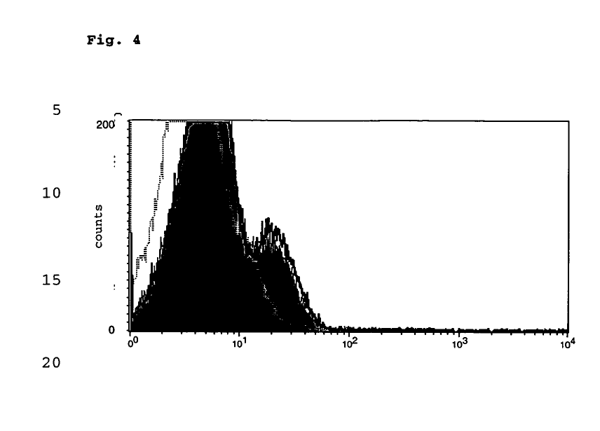

In contrast, preincubation of the mouse anti-human TLR9 mAb with the control peptide had no effect on TLR9 staining (FIG. 4). Because the TLR9 peptide and not an irrelevant peptide can block the fluorescence observed with the mouse anti-human TLR9 mAb, these data confirm the specificity of the TLR9 staining. Histograms shown are gated on live cells and show fluorescence for the mouse anti-human TLR9 mAb (gray histogram), the mouse anti-human TLR9 mAb preincubated with a control peptide (bold black line), and the mouse isotype-APC mAb (thin stippled line). Preincubation of mouse anti-human TLR9 mAb with a control peptide has no effect on the fluorescence staining observed with the TLR9 mAb. Together these data presented in FIGS. 3 and 4 confirm the specificity of the PBMC labeling observed with the TLR9 mAb and that the TLR9⁺ PBMC populations observed in the preceding Examples are not artifactual.

### EXAMPLE 6

### Increased TLR9 Transcript Levels in a Mouse Model of Chronic Lung Inflammation

Gene transcript levels, as assessed by real time PCR, are generally regarded by those of ordinary skill in the art as a proxy for gene (protein) expression levels. Real time-PCR was used to quantify TLR9 gene transcript levels in the lung tissues of SP-C/TNF-α transgenic and wild-type mice. SP-C/TNF-α transgenic mice overexpress TNF-α in alveolar type II cells. This TNF-α overexpression is controlled in these mice by the Human surfactant protein C promoter (Fujita et al., Am. J. Physiol.—Lung C 280:L39-49, (2001)). Histopathological studies have revealed chronic lung inflammation in SP-C/TNF-α transgenic mice. Additionally, physiological assessments have demonstrated that SP-C/TNF-α transgenic mice exhibit increased lung volumes and a decrease in elastic recoil characteristic of emphysema (Fujita et al., supra). The SP-C/TNF-α transgenic mice are an accepted mouse model for chronic lung inflammation.

Total RNA for real time-PCR analysis was extracted from mouse lung tissue samples using Trizol™ (Invitrogen Corp., Carlsbad, Calif.) according to the manufacturer's instructions. cDNAs were prepared using the Omniscript™ kit (Qiagen Inc., Valencia, Calif.) according to manufacturer's instructions. TaqMan™ real time-PCR was then performed in 50 ml volumes on 96-well plates using the Universal Master Mix (Applied Biosystems) and ABI PRISIM™ 7000HT instrumentation. The TaqMan™ real-time PCR technology and ABI instrumentation detect accumulation of PCR products continuously during the PCR process and allow accurate transcript quantitation in the early exponential phase of PCR. Primer Express™ software was used to design the probe sequence 5′-CGTCGCTGCGACCATGCC-3′ (SEQ ID NO: 5), the forward primer sequence 5′-ACTTGATGTGGGTGGGAATTG-3′ (SEQ ID NO: 6) and the reverse primer sequence 5′-GCCACATTCTATACAGGGATTGG-3′ (SEQ ID NO: 7). cDNA levels were normalized against transcipt levels for the thioredoxin reductase housekeeping gene. The thermal cycling protocol started with a 50° C. annealing step for two minutes, followed by ten minutes at 95° C. to denature the DNA and activate the AmpliTaq Gold™ polymerase. This was followed by 40 cycles of 95° C. for 15 seconds and 60° C. for one minute during which the AmpliTaq Gold™ polymerase cleaves the probe and the fluorescence data is collected. The data collection and transcript quanitation in the early exponential phase is performed by the ABI PRISIM™ 7000HT instrumentation and associated software.

The results are presented in Table 4 and show that TLR9 mRNA transcript levels are increased in the lung tissue of SP-C/TNF-α transgenic mice as compared to the lung tissue of age-matched, wild-type control mice. Peak levels of TLR9 mRNA expression were observed in 9 week-old transgenic mice, an age that correlates with a marked inflammatory response in the lungs (Fujita et al., supra). These data demonstrate that TLR9 transcript levels, and presumably expression, are increased in the lungs of SP-C/TNF-α transgenic mice. Lastly, the data indicates a role for TLR9 in TNF-α driven lung inflammation.

TABLE 4
Real time PCR quantitative analysis of TLR9 expression in
transgenic SP-C/TNF-α mice.

Age (weeks)

| | 4 | 6 | 9 | 14 |
|---|---|---|---|---|
| TLR9 fold | 1.2 | 3.22 | 5.23 | 2.88 |
| increase* | | | | |

*Relative fold increase in mRNA expression compared to a reference wild-type mouse given a value of 1. cDNA sample TLR9 levels were normalized against transcript levels of the thioredoxin reductase housekeeping gene.

### EXAMPLE 7

### Generation of Anti-TLR9 mAbs

Separate groups of mice will be immunized with plasmid DNA encoding the extracellular domain of TLR9 (residues 1 through 818 of SEQ ID NO: 1). Each mouse will receive three 15 μg doses of plasmid DNA diluted in PBS (150 mM NaCl; pH 7.4), each dose to be injected intradermally in the ears two weeks apart. After the plasmid DNA injections, mice will be boosted twice at biweekly intervals (15 μg per mouse injected intradermally) with a Fc fusion or mimetibody construct containing the 260 residue extracellular domain fragment of TLR9 or its mature form such as the fusion protein having the sequence shown in SEQ ID NO: 11. Spleens from immunized mice will be harvested and B cell fusions carried out using standard hybridoma methods of Kohler et al., supra). Three days prior to B cell fusion, mice will be given an intravenous injection of 15 μg of the protein used for boosting. Fused cells will be selected using HAT medium and will be screened for the presence of anti-TLR9 antibodies by ELISA. Fused cells testing positive will be expanded and cloned by limiting dilution. Anti-TLR9 antibody nucleic acid and protein sequences will be determined by standard techniques.

### EXAMPLE 8

### CpG-Dependent Binding of EC260-Fc to CpG Oligodinucletotide

A human TLR9 extracellular domain Fc fusion construct was made as follows. A cDNA fragment encoding amino acids 1 to 260 of human TLR9 was amplified by polymerase chain reaction and cloned into a FcHA6His-Fly FLY cell expression vector resulting in a fusion protein containing the first 260 amino acids of TLR9 fused in-frame with an Fc fragment of human IgG1 followed by a hemagglutinin tag and a hexa-histidine tag at the C-terminus. The complete coding sequence of the fusion protein was then excised and cloned into a pcDNA3.1/(+) vector (Invitrogen, Carlsbad, Calif.) at the EcoR1 and Xho1 sites.

HEK293 cells were transfected with the pcDNA3.1/(+)-EC260-FcHAhexaHis vector and selected in 400 μg/ml G418. The EC260-Fc protein was detectable from cells and as a soluble dimer protein in culture supernatant. The amino acid sequence of the fusion protein construct is shown in SEQ ID NO: 2. The secreted form of the fusion protein will lack the signal sequence and is predicted to have the amino acid sequence shown in SEQ ID NO: 11.

Culture supernatant from stable HEK293 cells expressing and secreting EC260-Fc was harvested and cleared by centrifugation. Protein A-Sepharose (PAS) beads (Amersham Biosciences, Piscataway, N.J.) were added to the cleared supernatant and incubated at 4° C. for >2 hours to allow for binding of the EC260-Fc construct to the beads. After incubation, the beads were pelleted by centrifugation and washed twice with saline.

The synthetic immunostimulatory CpG oligodinucleotide (ODN) ODN2006 (SEQ ID NO: 8) was end-labeled with ³³P using T4 kinase (Promega, Madison, Wis.) and ³³P-γ-ATP (Amersham Biosciences, Piscataway, N.J.). The end-labeled ODNs were then separated from free ³³P-γ-ATP by G25 (Amersham Biosciences, Piscataway, N.J.) column chromatography.

The CpG-dependent binding of ODNs by PAS-bound EC260-Fc protein was examined by incubating PAS-EC260-Fc beads with ³³P-ODN2006 in binding buffer (10 mM Tris.HCl, pH6; 50 mM NaCl, 1 mM MgCl, 0.5 mM EDTA, 1 mM DTT, 0.1% NP-40, 0.03% BSA, 5% Glycerol) in the presence of excess non-specific DNA from salmon testes (50 μg/ml) at room temperature for 2 hours followed by washing three times with saline. Bead-bound radioactivity was determined in a TopCount scintillation counter (PerkinElmer, Boston, Mass.). To determine if binding is CpG dependent, separate binding reactions were run including a 50-fold excess of unlabeled ODN2006 or an inactive oligo where the CpG dinucleotides of ODN2006 were changed to GpC dinucleotides (ODN2006GC, SEQ ID NO: 9) or the CpG to GpC changes were made in addition to changes in the flanking sequences (SEQ ID NO: 10.

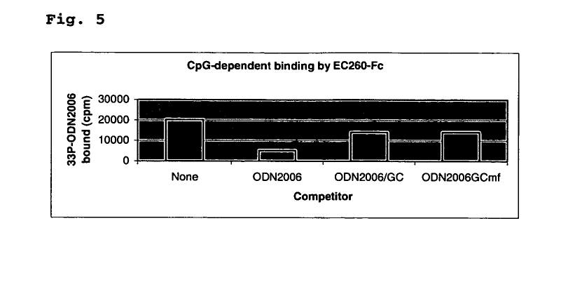

The results shown in FIG. 5 indicate that a significant amount of ³³P-ODN2006 was detected on the PAS-EC260-Fc beads. The results suggest that the binding is CpG-dependent since ODN2006 competed well for binding while the inactive oligos ODN2006-GC and ODN2006GCmf did not.

### EXAMPLE 9

### Effect of EC260-Fc on CpG-Induced Cytokine Production

An extracellular domain fragment of hTLR9 (residues 1 to 260) generated using a transient transfection protocol was tested for its ability to compete with the TLR9 ligand CpG ODN for cytokine production in human PBMCs. Human PBMC secrete a variety of cytokines and chemokines including IFN-γ, IFN-α, TNF-α, IL-10, IL-12, IL-8, MCP-1, MIP1-α and RANTES in response to CpG stimulation. Human PBMCs were isolated using standard Ficoll gradient and stimulated with either CpG alone or with CpG pre-incubated for 1 hour at 37° C. with the mature form of the 1 to 260-Fc fusion protein domain of human TLR9 (SEQ ID NO: 11). Culture supernatants were harvested at 24 hours after stimulation and cytokine levels were analyzed using Luminex.

The percent inhibition in cytokine production observed when the CpG-ODN were pre-cultured with the TLR9-Fc reagent prior to the addition to PBMC cultures, relative to CpG-ODN stimulation alone is shown in the Table 5 below. The results indicate that the human TLR9 domain consisting of amino acids 26-260 is capable of interfering with CpG-induced IFN-γ, IL-10, IL-6, MCP-1, MIP1-α, RANTES and TNF-α production, indicating that the TLR9-Fc fusion protein could serve as a sink to minimize the ability of bacterial CpG to stimulate the secretion of inflammatory cytokines during bacterial infections.

TABLE 5
Percent inhibition of cytokine production compared to CpG stimulation alone.

| | IFNγ | IFNα | IL10 | IL12 | IL1b | IL6 | IL8 | MCP-1 | MIP1α | RANTES | TNFα |
|---|---|---|---|---|---|---|---|---|---|---|---|
| 24 hr | 65% | 0% | 70% | 0% | 0% | 49% | 0% | 24% | 49% | 33% | 41% |

The present invention now being fully described, it will be apparent to one of ordinary skill in the art that many changes and modifications can be made thereto without departing from the spirit or scope of the appended claims.

1. A method of modifying antigen presenting cell function in a patient in need thereof comprising administering to the patient a cell surface TLR9 binding agent that specifically binds to human TLR9 in an amount effective to modify antigen presenting cell function in the patient.

2. The method of claim 1 wherein the binding agent is a human TLR9 antagonist antibody or fragment thereof.

3. The method of claim 1 wherein the binding agent is a human TLR9 agonist antibody or fragment thereof.

4. A method of identifying TLR9 binding agents comprising the steps of:
a. contacting MHCII⁺CD19⁺ or MHCII⁺CD19⁻ primary human cells expressing TLR9 on their surface with a putative binding agent;
b. measuring the binding of the putative binding agent to the cell surface and the effect on TLR9 biological activity; and
c. identifying TLR9 binding agents affecting TLR9 biological activity.

5. A method of modifying antigen presenting cell function in a patient in need thereof comprising administering to the patient a TLR9 ligand binding agent that specifically binds to human TLR9 ligand in an amount effective to modify antigen presenting cell function in the patient.

6. The method of claim 5 wherein the TLR9 ligand is a CpG oligodinucleotide.

7. The method of claim 5 wherein the TLR9 ligand binding agent comprises residues 1 to 260 of the extracellular domain of human TLR9 protein, a fragment thereof or a mature form.

8. The method of claim 5 wherein the TLR9 ligand binding agent comprises residues 1 to 260 of the extracellular domain of human TLR9 protein, a fragment thereof or a mature form fused to a fusion partner.

9. The method of claim 8 wherein the fusion partner is an Fc region from an immunoglobulin molecule.

10. The method of claim 8 wherein the TLR9 ligand binding agent has the amino acid sequence shown in SEQ ID NO: 2.

11. The method of claim 8 wherein the TLR9 ligand binding agent has the amino acid sequence shown in SEQ ID NO: 11.

12. A TLR9 ligand binding agent comprising residues 1 to 260 of the extracellular domain of human TLR9 protein, a fragment thereof or a mature form.

13. A TLR9 ligand binding agent comprising residues 1 to 260 of the extracellular domain of human TLR9 protein, a fragment thereof or a mature form fused to a fusion partner.

14. The TLR 9 ligand binding agent of claim 13 wherein the fusion partner is an Fc region from an immunoglobulin molecule.

15. A TLR9 ligand binding agent having the amino acid sequence shown in SEQ ID NO: 2.

16. A TLR9 ligand binding agent having the amino acid sequence shown in SEQ ID NO: 11.

17. A method of identifying TLR9 binding agents comprising the steps of:
a. contacting the TLR9 ligand binding agent of claim 14 with a putative TLR9 binding agent;
b. measuring the binding of the putative TLR9 binding agent to the TLR9 ligand binding agent and the effect on TLR9 biological activity; and
c. identifying TLR9 binding agents affecting TLR9 biological activity.


SEQUENCE LISTING (11 sequences)

SEQ ID NO: 1 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 2 - protein, 514 aa, Artificial
  TLR9 1-260 Fc Fusion
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIDRSYPYDVPDYARSHHHHHH

SEQ ID NO: 3 - protein, 17 aa, Homo sapiens
CPRHFPQLHPDTFSHLS

SEQ ID NO: 4 - protein, 15 aa, Homo sapiens
CEKHSQPWQVLVASR

SEQ ID NO: 5 - DNA, 18 bp, Mus musculus
cgtcgctgcgaccatgcc

SEQ ID NO: 6 - DNA, 21 bp, Mus musculus
acttgatgtgggtgggaattg

SEQ ID NO: 7 - DNA, 23 bp, Mus musculus
gccacattctatacagggattgg

SEQ ID NO: 8 - DNA, 24 bp, Artificial
  Oligodinucleotide
tcgtcgttttgtcgttttgtcgtt

SEQ ID NO: 9 - DNA, 24 bp, Artificial
  Oligodinucleotide
tgctgcttttgtgcttttgtgctt

SEQ ID NO: 10 - DNA, 24 bp, Artificial
  Oligodinucleotide
tcttgcgtttttgcgtttttgcgt

SEQ ID NO: 11 - protein, 489 aa, Artificial
  Predicted Mature Form of TLR9 EC260-Fc Fusion
LGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIDRSYPYDVPDYARSHHHHHH